(19) United States
(12) Patent Application Publication
(10) Pub. No.: US 2021/0356149 A1
(43) Pub. Date: Nov. 18, 2021

(54) CLEAN AIR SYSTEM FOR CASINO TABLE GAMES, SLOT MACHINES AND OTHER APPLICATIONS

(71) Applicant: David WARREN, Stone Ridge, NY (US)

(72) Inventor: David WARREN, Stone Ridge, NY (US)

(21) Appl. No.: 17/319,344

(22) Filed: May 13, 2021

Related U.S. Application Data
(60) Provisional application No. 63/024,973, filed on May 14, 2020.

(51) Int. Cl. F24F 8/22; F24F 5/00; F24F 8/80; F24F 13/06; A63F 3/00; A61L 2/10

(52) U.S. Cl. CPC F24F 8/22; F24F 5/0096; F24F 8/80; F24F 13/0604; A61L 2209/16; A61L 2/10; F24F 2221/10; A63F 2003/00164; A63F 3/00157

(57) ABSTRACT

A clean air system comprises an inlet port, an airflow chamber, and an air exit port. A UV light source arranged within the airflow chamber disinfects air as it passes through the airflow chamber. The chamber promotes a vortex of swirling air around the intense UV light maximizing particle exposure. The clean air is then expelled at the other end of the chamber.

### CROSS-REFERENCE TO RELATED APPLICATIONS

This application claims priority to U.S. Provisional Application Ser. No. 63/024,973, filed May 14, 2020, the entire contents of which are hereby incorporated by reference in their entirety.

### TECHNICAL FIELD

The present invention relates to systems and devices to provide clean air for casino table games, slot machines, and other applications to, e.g., protect dealers, other employees, and customers from airborne contaminants and hazards, such as bacteria, viruses, and other germs.

### BACKGROUND

With the current COVID-19 pandemic, the threat of airborne contaminants and hazards is all the more pertinent. These threats are relevant during the pandemic and during the recovery or return period thereafter. More notably, however, is the effect the pandemic has had on raising everyone's awareness of the threats and dangers inherent in public settings.

Such threats and dangers are perhaps nowhere more visible and real than in the modern casino. Social distancing is practically impossible for anyone in a casino to practice, least of all the dealers and pit-bosses that work at the various table games. When standing or sitting directly across from hundreds or thousands of different casino patrons a day, such casino employees are directly at risk of exposure to such contaminants and hazards.

There exists systems that produce air curtains or barriers to protect dealers. See, e.g., U.S. Pat. No. 6,210,267, which is hereby incorporated by reference in its entirety. However, such systems do not adequately protect dealers from biological airborne contaminants such as bacteria, virus, and fungi.

What is needed then is a device that protects such dealers, pit-bosses, and other casino workers from airborne contaminants and hazards, directly addressing the problems described above.

### SUMMARY

Embodiments of the present invention may provide a clean or pure air system. A challenge is to provide a safe clean air environment for the dealer and players at the gaming table without disrupting play.

Embodiments clean the air circulating above a table using ultraviolet (UV) light, e.g., encased by a cylinder (for example, stainless steel metal tubing). The encasing protects participants from the radiation of a UV light, while the tubing helps circulate air effectively. This system works by having a fan pushing or pulling untreated air around a UV light chamber. The chamber promotes a vortex of swirling air around the intense UV light maximizing particle exposure. The clean air is then expelled at the other end of the chamber.

Embodiments of the clean air system kill bacteria, fungus, and/or viruses and cleans the air without disrupting airflow or requiring a filter. The contaminated air is pulled and then passes the UV light chamber before being expelled at the other side of the table.

UV light for many years has been used for disinfecting bacteria from potable water and killing mold and other fungi in heating, ventilation, and air-conditioning (HVAC) systems.

Water-Treatment Applications:

UV disinfection deactivates living organisms, but it does not remove particles from water, add chemicals, or remove bad tastes and odors. UV disinfection has been used to treat water for: Cryptosporidium, Giardia, Dysentery bacilli, Salmonella, Mycobacterium tuberculosis, Streptococcus, E. coli, Hepatitis B, Cholera, Algae, Fungi and some viruses.

Air-Treatment Applications:

Indoor air quality is a critical factor for the health of any individual or family. According to the Environmental Protection Agency (EPA), indoor air is often five (5) times more polluted than outdoor air. UV clean air products improve indoor air quality throughout a home or office by cleaning the air as it passes through the central HVAC system. UV clean air products also improve the efficiency and longevity of a system by keeping it clean and free of mold.

Recent studies and articles have shown the efficacy of UV light at killing the COVID-19 and other viruses. See, for example, “Could a New Ultraviolet Technology Fight the Spread of Coronavirus?,” Columbia News by Carla Cantor, Apr. 21, 2020 (https://news.columbia.edu/ultraviolet-technology-virus-covid-19-UV-light), which describes Professor David Brenner's research team's experiments showing far-UVC light's efficacy at eradicating two types of airborne seasonal coronavirus. Other articles also describe UV light's, and particularly UVC light's, efficacy at killing and/or inactivating coronaviruses, influenza, and other virus, including COVID-19; see, e.g., “Can you kill coronavirus with UV light?,” BBC.com Future by Zaria Gorvett, Apr. 24, 2020 (https://www.bbc.com/future/article/20200327-can-you-kill-coronavirus-with-uv-light), “Can UV Light Fight the Spread of Influenza,” Columbia University Irving Medical Center, Feb. 9, 2018 (https://www.cuimc.columbia.edu/new/can-uv-light-fight-spread-influenza), and “Could ultraviolet lamps slow the spread of flu?,” Science by Jon Cohen, Jan. 3, 2018 (https://www.sciencemag.org/news/2018/01/could-ultraviolet-lamps-slow-spread-flu#).

### BRIEF DESCRIPTION OF THE DRAWINGS

Embodiments of the present invention are illustrated by way of example, and not limitation, in the accompanying figures in which like references denote similar elements, and in which:

FIG. 1 is a cross-sectional, side view diagram of an embodiment of a clean air system installed on a casino table game.

FIGS. 2A-2F are top view diagrams of different embodiments of a clean air system installed on a casino blackjack or similar table game.

FIGS. 3A-3B are cross-sectional side views of embodiment of a clean air system installed in a player rail of a casino table game.

FIGS. 4A-4B are cross-sectional view diagrams of an ultraviolet light bulb airflow chamber utilized by embodiments of a clean air system.

FIGS. 5A-5B are cross-sectional view diagrams of an ultraviolet LED airflow chamber utilized by embodiments of a clean air system.

FIG. 6 is a cross-sectional view diagram of a multiple ultraviolet light airflow chamber utilized by embodiments of a clean air system.

FIGS. 7A-7C are cross-sectional view diagrams of embodiments of a cone-shaped ultraviolet light airflow chamber utilized by embodiments of a clean air system.

FIG. 8 is side-view diagram of an embodiment of a clean air system for use with a casino table game.

FIG. 9 is a side view diagram of an embodiment of a clean air system for use with a slot or video game machine.

FIG. 10 is a side view diagram of an embodiment of a clean air system for use with a slot or video game machine.

FIG. 11 is a perspective view of a casino blackjack table with an embodiment of a clean air system.

### DETAILED DESCRIPTION

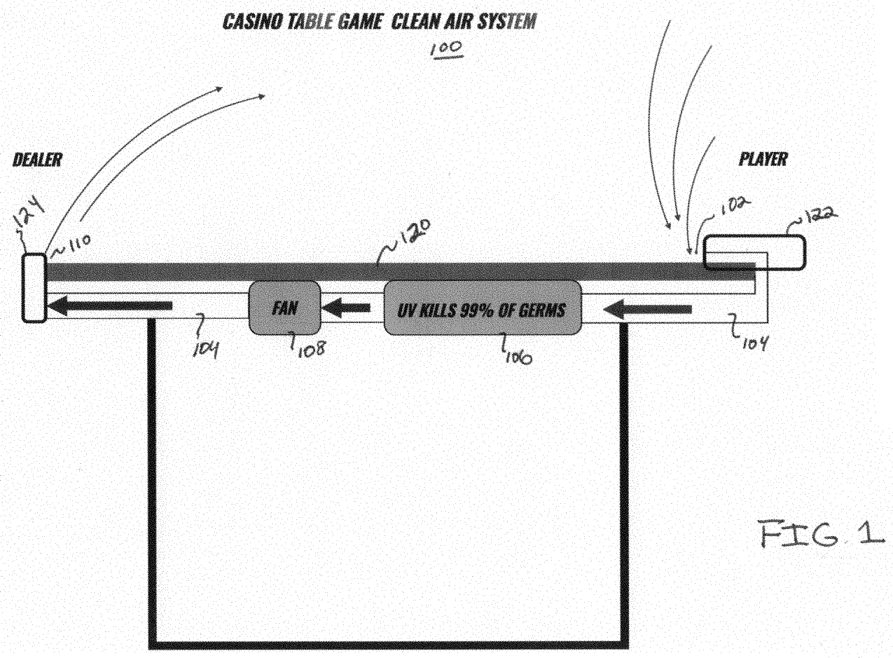

With reference now to FIG. 1, shown is cross-sectional diagram of an embodiment of a clean air system 100 installed on a casino table game. Clean air system 100 includes a dirty air intake/inlet 102, air channel 104, UV chamber 106, fan 108, and a clean air outlet 110. Fan 108 operates to draw “dirty” air through dirty air intake 102, through air channel 104 and UV chamber 106. UV lights, e.g., UVC lights, in UV chamber 106 treat dirty air received from dirty air intake 102, killing or de-activating bacteria, fungi, and viruses carried by dirty air, producing “clean” air. Fan 108 draws clean air out of UV chamber 106 through fan 108 and air channel 104 from which it exits through clean air outlet 110. Clean air system 100 may be installed on or under casino gaming table 120 so that dirty air intake 102 is located near, e.g., in front of, players sitting or standing at rail 122 and clean air outlet 110 is located near, e.g., in front of, dealer standing or sitting at dealer rail or side 124 of table. Dirty air intakes 102 may be placed in table surface in front of rail 122 or directly in rail 122. Likewise, clean air outlets 110 may be placed into table surface in front of dealer rail or side 124 of table (or, e.g., in dealer rail 124). In this manner, air exhaled by players, which may contain contaminants such as bacteria, fungi, and viruses, is drawn downward, away from player (and dealer), is cleaned, and resulting clean air is blown in front of and away from dealer. In addition to having had contaminants de-activated or killed, the blown clean air also forms an air curtain or barrier that helps to prevent dirty air from being blown across the table towards dealer.

As noted above, dirty air intake may be located in front of players sitting or standing at rail of casino table game table. Likewise, clean air outlet may be located in front of dealer standing or sitting at dealer rail or side of casino table game table. Embodiments may also include multiple dirty air intakes and multiple clean air outputs.

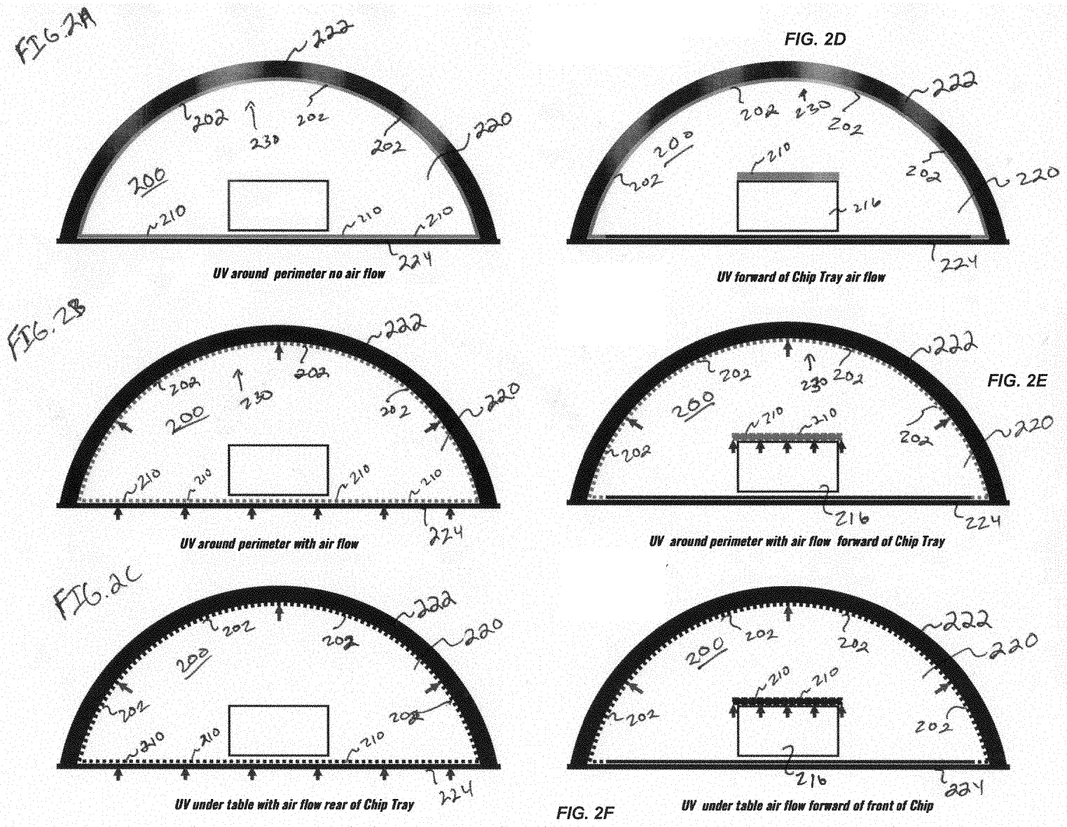

With reference now to FIGS. 2A-2F, shown are top views of multiple embodiments of a clean air system 200 installed on a casino table game. Shown in FIGS. 2A-2C are embodiments of a clean air system 200 which includes multiple dirty air intakes 202 located around perimeter of player rail 222 of gaming table 220. In such embodiments, dirty air intakes 202 may be closely spaced and placed continuously around perimeter of player rail 222, as shown (e.g., in front of or in player rail 222). Likewise, multiple clean air outlets 210 may be placed at dealer rail or side 224 of gaming table. Indeed, clean air outlets 210 may be closely spaced and placed continuously along at dealer rail or side 224.

In embodiment shown in FIG. 2C, clean air system 200 may be configured as shown in FIG. 1, with air channel or channels (not shown) connecting dirty air intakes 202 with UV chamber (or chambers) (not shown), which treats dirty air received from dirty air intakes 202, producing clean air that is pulled or sucked through UV chamber by fan (not shown) and expelled through clear air outlets 210. Air channel, fan and UV chamber(s) may be installed underneath the gaming table 220 (e.g., affixed to the bottom side of table 220 or freestanding, separate equipment). In embodiments, there may be multiple feeder air channels feeding dirty air to a single UV chamber, creating a stronger air flow through volume reduction. Additionally, clean air outlets 210 may have valves or covers that allow dealer to selectively close clean air outlets 210, producing a smaller but strong “air curtain” in front of dealer.

As shown in FIGS. 2A-2C, instead of a UV chamber(s) affixed to the underside of the gaming table, clean air system 200 may include UV light sources located in the player rail 222 (e.g., near each dirty air intake 202 and/or each clean air outlet 210). In this manner, dirty air may be treated by UV lights at each dirty air intake 202 and/or clean air outlet 210. In such an embodiment, UV light emitted by UV light source may create a UV glow from the air intakes 202 and air outlets 210. FIG. 2A shows clean air system 200 without air flow, illustrating UV light 230 glow emitted by UV light sources around perimeter of table 220. Shielding (not shown) installed in player rail 222 shields players from harmful effects of UV light, permitting only a harmless UV light 230 glow to be emitted. Air channel and fan, connecting dirty air intakes 202 with clean air outlets 210, may be installed in player rail 222 or underneath table 220, as in FIG. 2C.

With reference now to FIGS. 2D-2F, shown are embodiments similar to the embodiments shown in FIGS. 2A-2C, except that clean air outlets 210 are located in front of chip tray 216 (in FIG. 2F, UV chamber is located underneath table 220). Since dealer stands directly behind chip tray 216, such an location for clear air outlets 210 is sufficient to protect dealer. Clean air outlets 210 may be closely spaced and placed continuously along front side of chip tray 216. Combinations of the embodiments shown in FIGS. 2A-2C with the embodiments shown in FIGS. 2D-2F may also be implemented with clean air outlets 210 placed along front side of chip tray 216 and along dealer rail or side 224 to left and right of chip tray 216.

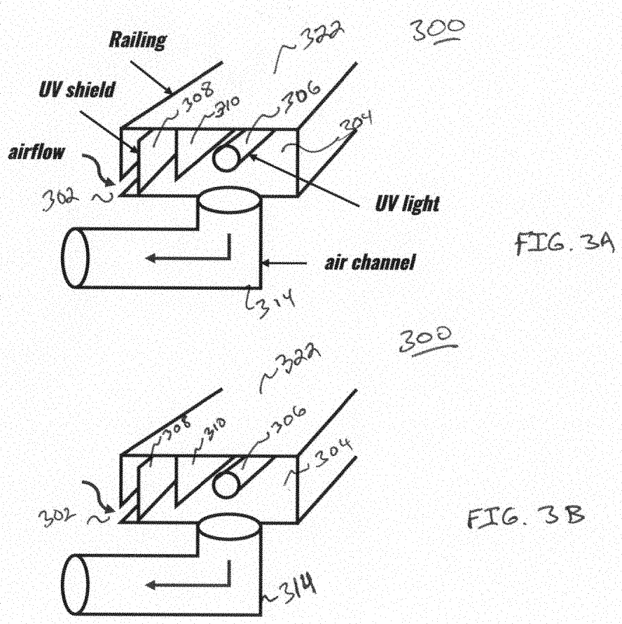

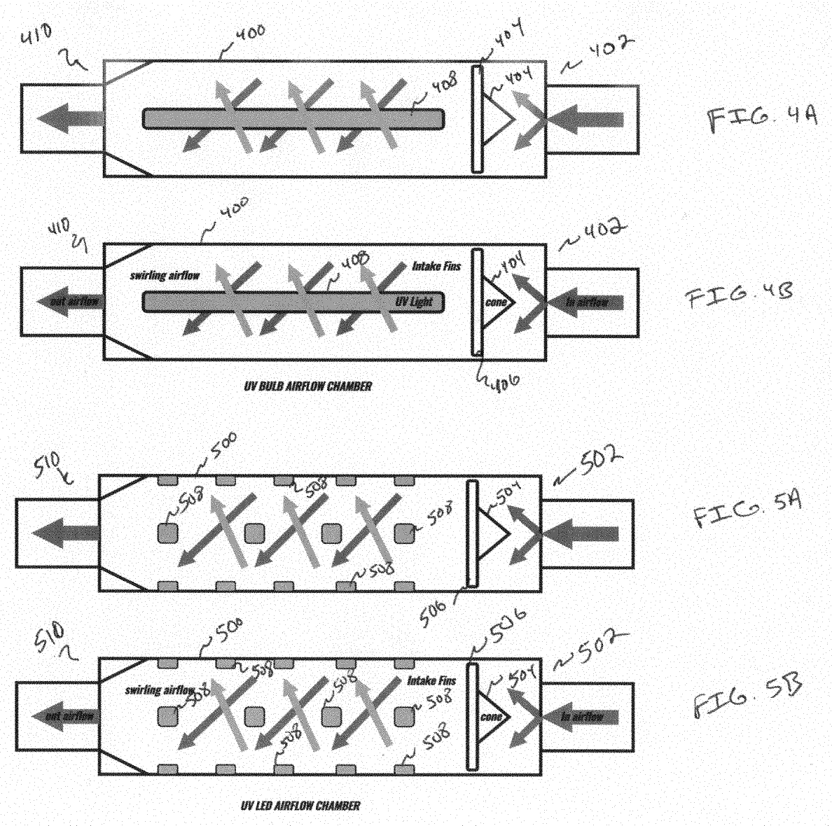

With reference now to FIGS. 3A-3B, shown is an embodiment of clean air system 300 in which UV light source is placed within player rail. Clean air system 300 may be used in embodiments shown in FIGS. 2A-2B and 3A-3B in which dirty air intakes 202 are placed in front of or along player rail 222. In embodiment shown, dirty air intake(s) 302 is placed along front of player rail or railing 322. Dirty air intake 302 may be a continuous opening along front of player rail 322. Alternatively, multiple dirty air intakes 302 may be formed as spaced openings in along front of player rail 322. Dirty air is drawn into dirty air intake 302 into interior of rail 322 by fan (not shown) which may be placed in rail 322 or connected to air channel 314 installed underneath gaming table (not shown). Interior of player rail 322 may be substantial hollow, forming a chamber or channel 304 along length of player rail 322. Dirty air may flow into chamber formed in interior of player rail 322 where it is exposed to UV light generated by UV light source 306 placed in chamber or channel 304. UV light source 306 may be a UV light bulb or bulbs or UV light emitting diodes (LEDs). UV light source 306 may be multiple bulbs or LEDs. UV light source 306 may emit UVC light of sufficient bandwidth (e.g., in an embodiment, bandwidth may be UVC 100 nm to 280 nm) to kill or de-activate 99.9% of known contaminant. Chamber or channel 304 formed by railing 322 may also include UV shield 308 to shield or prevent harmful UV light from being emitted from railing 322. Chamber 304 may also include intake fin(s) or blade(s) 310 which act to impart a spin or vortex on dirty air flow, maximizing exposure to UV light generated by UV light source 306. UV light source 306 may generate UVC light. Clean air produced by exposure of dirty air to UV light may exit chamber 304 and drawn or sucked through air channel 314 towards clean air outlets (not shown).

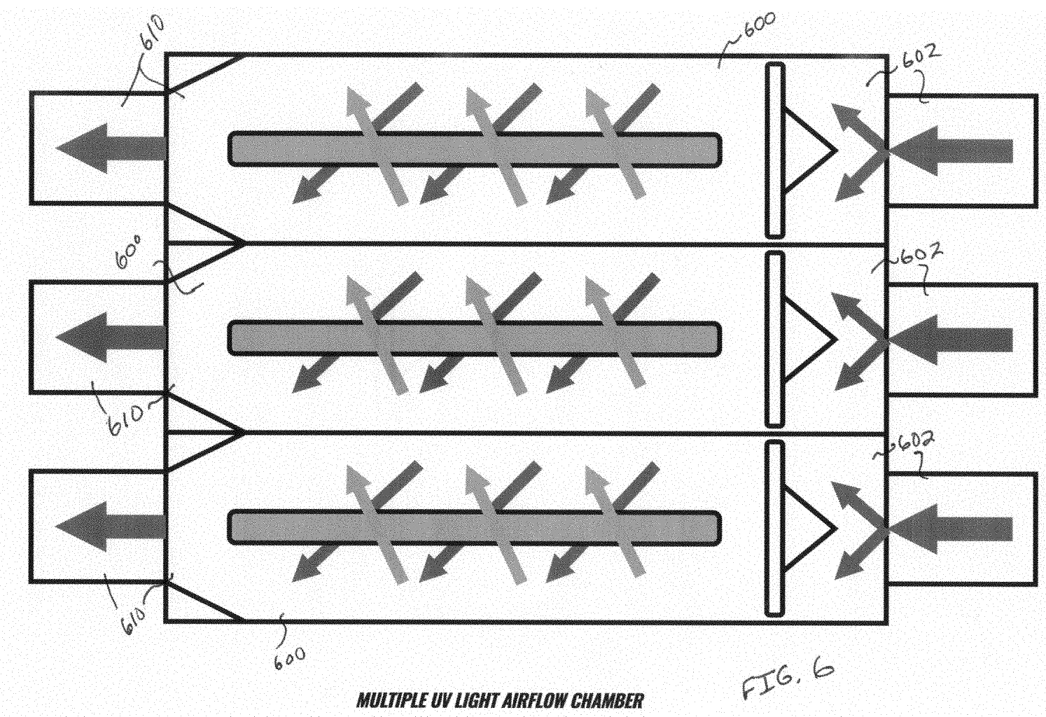

With reference now to FIGS. 4A-4B, shown is an embodiment of UV chamber or UV airflow chamber 400. UV chamber 400 may be used as the UV chamber 106 shown in FIG. 1 or the UV chambers described with reference to FIGS. 2C and 2F. UV chamber 400 includes an dirty air intake end 402, a cone 404, intake fins 406, UV light bulb 408, and a clean air outlet 410. UV light bulb 408 may emit UVC light of sufficient bandwidth (e.g., in an embodiment, bandwidth may be UVC 100 nm to 280 nm) to kill or de-activate 99.9% of known contaminants. Fan (not shown) draws dirty air through dirty air intake 402. The dirty air flows over cone 404 and intake fins 406 which causes air to spin or swirl around inside of UV chamber 400, as shown by directional arrows. Cone 404 and intake fins 406 are sized, shaped, placed, and configured to impart the spin or swirl on the air flow (a vortex). The swirling air flow or vortex maximizes the exposure of the dirty air and the contaminants it contains to the UV light emitted by the UV light bulb 408. This, in turn, maximizes the efficacy of the UV light in killing or de-activating the bacteria, fungi, and viruses in the dirty air flow. Air in which contaminants have been killed or de-activated, i.e., clean air, is drawn through and output through clean air outlet 410. UV chamber 400 may be configured as a cylinder or other shape and is preferably made from a material that prevents any air or UV light leakage (e.g., plastic, stainless steel metal tubing, etc.).

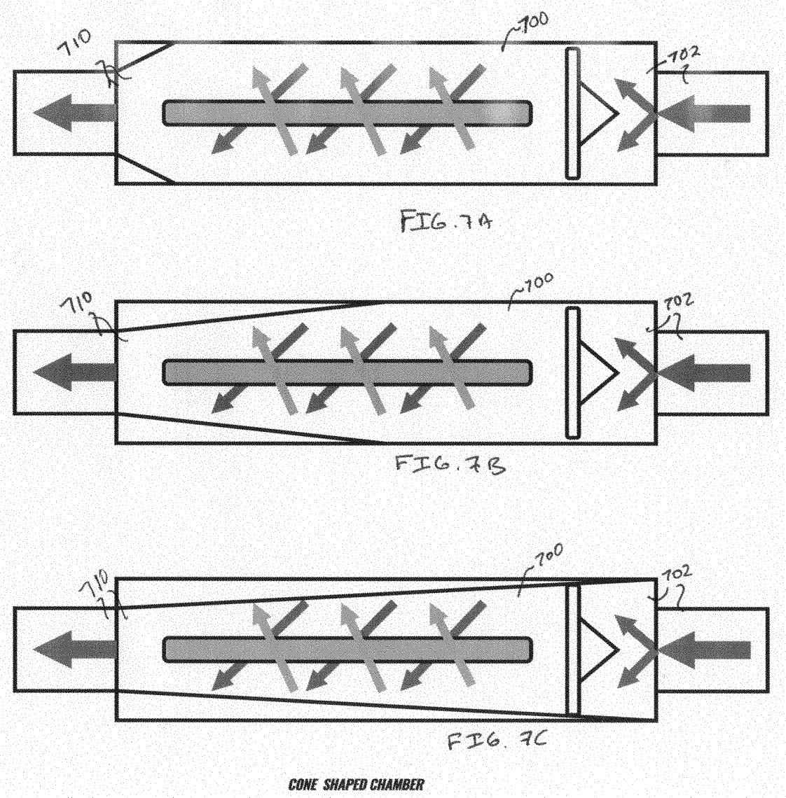

With reference now to FIGS. 5A-5B, shown is another embodiment of UV chamber or UV airflow chamber 500. Like the embodiment shown in FIGS. 4A-4B, UV chamber 500 may be used as the UV chamber 106 shown in FIG. 1 or the UV chambers described with reference to FIGS. 2C and 2F. UV chamber 400 includes an dirty air intake end 502, a cone 504, intake fins 506, multiple UV light-emitting diodes (LEDs) 508, and a clean air outlet 510. UV LEDs 508 may emit UVC light of sufficient bandwidth (e.g., in an embodiment, bandwidth may be UVC 100 nm to 280 nm) to kill or de-activate 99.9% of known contaminants. UV LEDs 508 may be placed around and throughout interior of UV chamber 500 (e.g., along interior wall of chamber 500 and/or suspended in center of chamber 500). As in embodiment described above, fan (not shown) draws dirty air through dirty air intake 502. The dirty air flows over cone 504 and intake fins 506 which causes air to spin or swirl around inside of UV chamber 500, as shown by directional arrows. Cone 504 and intake fins 506 are sized, shaped, placed, and configured to impart the spin or swirl (creating a vortex) on the air flow. The swirling air flow maximizes the exposure of the dirty air and the contaminants it contains to the UV light emitted by the UV LEDs 508. This, in turn, maximizes the efficacy of the UV light in killing or de-activating the bacteria, fungi, and viruses in the dirty air flow. Air in which contaminants have been killed or de-activated, i.e., clean air, is drawn through and output through clean air outlet 510. UV chamber 500 may be configured as a cylinder or other shape and is preferably made from a material that prevents any air or UV light leakage (e.g., stainless steel metal tubing).

Embodiments of clean air system may include a single or multiple UV chambers. Shown in FIG. 6 is an embodiment of a clean air system that includes multiple UV chambers 600. UV chambers 600 may each have their own dirty air intake 602, connected to different air channels (not shown), and own clean air outlet 610, connected to different air channels, as shown, receiving dirty air from different dirty air intakes on gaming table (not shown) and outputting clean air to different clean air outlets (not shown). Alternatively, UV chambers may receive dirty air from a single dirty air intake and/or air channel, and output to a single clean air output and/or air channel. UV chambers 600 may each have UV light bulb(s) as shown or UV LEDs.

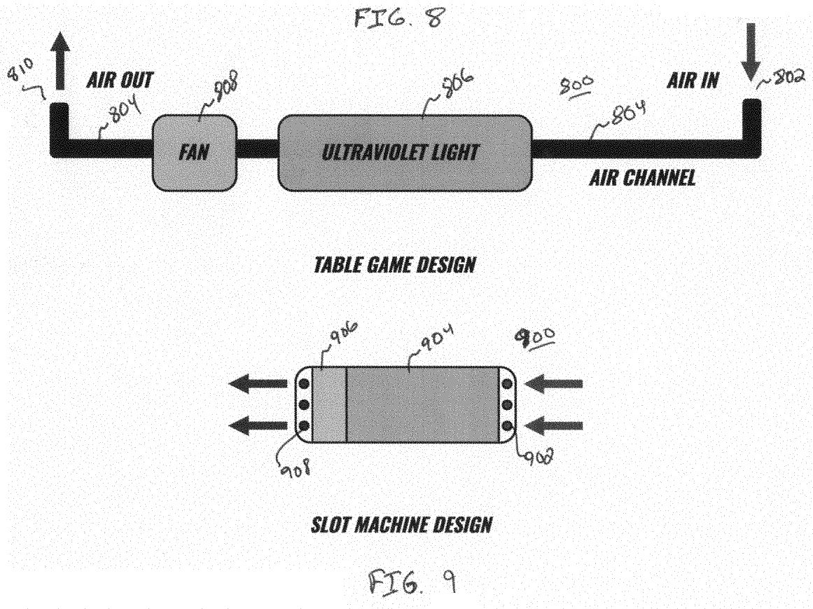

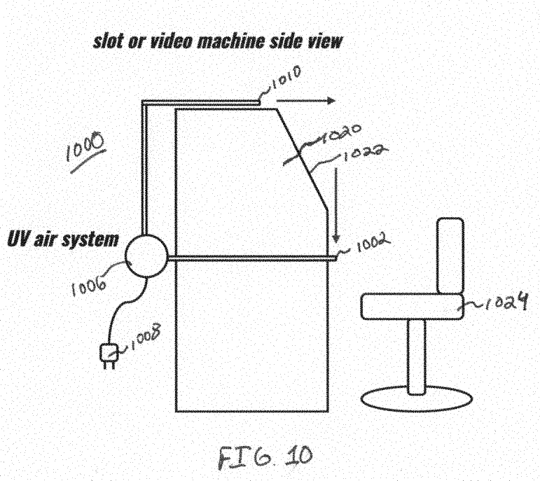

With reference now to FIGS. 7A-7C, shown are different embodiments of UV chamber 700. As shown, interior of UV chamber 700 may narrow from dirty air intake end 702 to clean air outlet end 710. In each embodiment shown progressing from FIG. 7A to 7C, the narrowing of interior of UV chamber 700 is more pronounced. This narrowing results in a more cone-like interior of UV chamber 700 with the volume of UV chamber reducing from the intake end 702 to the outlet end 710. This volume reduction increases the speed of the air flow through the UV chamber 700 and creates a stronger vortex (stronger spin).

With reference now to FIG. 8, shown is an embodiment of clean air system 800 for installation on a table game. Dirty air is drawn into system 800 through dirty air intake 802 into air channel 804 and through UV light chamber 806 by fan 808. Clean air is output from UV light chamber 806 through remainder if air channel 804 and output through clean air outlet 810. Embodiments of clean air system 800 are installed underneath gaming table (not shown). In alternative embodiments, air channel may be flexible house or tubing that connects to a portable UV chamber and which may be installed and removed from a gaming table. Flexible air channel may connect to air intakes and air outlets that are installed in gaming table either permanently or temporarily.

With reference now to FIG. 9, shown is a slot or video game machine embodiment of clean air system 900. Clean air system 900 includes dirty air intakes 902 connected directly to UV chamber 904. Fan 906 is connected directly to or placed within clean end of UV chamber and draws dirty air from dirty air intakes 902 directly into UV chamber 904. UV light (e.g., UVC light) in UV chamber 904 treats dirty air and produces clean air, which is drawn by fan 906 and output or expelled through clean air outlets 908 that are directly connected to clean air/output end of UV chamber 904. Video screen (not shown) of slot machine/video game machine may be placed immediately in front of UV chamber 904 and fan 906. In an embodiment, orientation of clean air system 900 may be with dirty air intakes 902 at bottom of video/slot game machine screen and clean air outlets 908 at top of video/slot game machine screen.

With reference now to FIG. 10, shown is an embodiment of a clean air system 1000 installed on a slot or video game machine 1020. As shown, dirty air intake(s) 1002 is in front, customer facing side of machine 1020 and may be installed below screen 1022 of slot or video game machine at or near typical face level of customer sitting on chair 1024. Air channel(s) 1004 may take dirty air drawn into dirty air intake(s) 1002 by fan (not shown) connected to air channel 1004. Dirty air flows through air channel(s) 1004 (e.g., located in interior (underneath screen and necessary electronics) or on exterior of slot or video game machine 1020. Air channel(s) 1004 is connected to and in communication with UV air system 1006, which may include fan(s) and UV chamber(s) (e.g., configured as described above). UV air system 1006 may include power cord or other power supply 1008. Indeed, UV chambers, light sources, and fans described above include power supplies configured to provide sufficient power to operate clean air systems described here, as is well within the skill of one of ordinary skill in the art. UV air system 1006 treats dirty air with UV light (e.g., UVC) of bandwidth capable of killing or de-activating 99.9% of known contaminants (e.g., in an embodiment, bandwidth may be UVC 100 nm to 280 nm). Clean air exits through remaining air channel(s) 1004 and is expelled through clean air outlet 1010. This blows clean air towards customer.

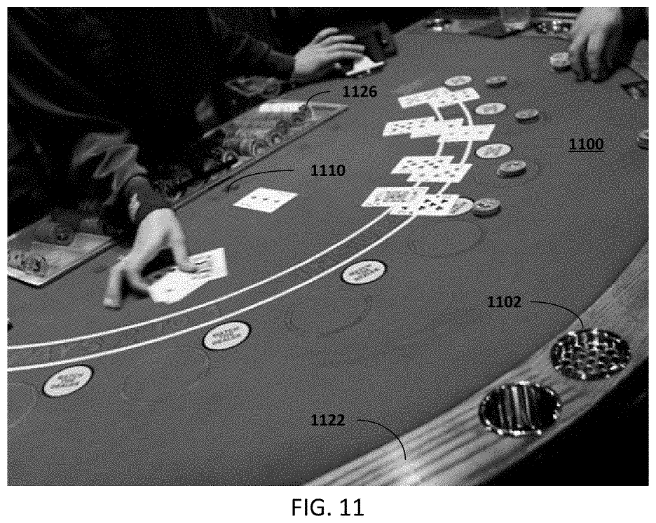

With reference now to FIG. 11, shown is a photograph illustrating an embodiment of a clean air system 1100 installed on a black jack table 1120 with dirty air intakes 1102 shown placed directly into player rail 1122 and clean air outlets 1110 placed into gaming table 1120 surface in front of chip tray 1126.

The clean air systems described herein can be installed on many different types of tables, bars, video screens and other applications. For example, clean air systems can be installed on bars to protect patrons and bartenders from air contaminants. Clean air systems can be installed in check-out, deli, and other counters at retail stores, take-out restaurants, food trucks, etc.

Several embodiments of the present invention are specifically illustrated and/or described herein. However, it will be appreciated that modifications and variations of the present invention are covered by the above teachings without departing from the spirit and intended scope of the invention.

1. A clean air system comprising:
a dirty air inlet port;
a clean air outlet port; and
an airflow chamber, wherein the airflow chamber comprises:
an air diffuser arranged in the airflow chamber proximal to the dirty air inlet port;
a plurality of intake fins located downstream and adjacent the air diffuser; and
an ultraviolet (UV) light source arranged downstream from the intake fins.

2. The clean air system according to claim 1, further comprising:
a fan located within or adjacent the clean air outlet port for drawing dirty air into the dirty air inlet port and through the airflow chamber.

3. The clean air system according to claim 1,
wherein the air diffuser has a cone shape, and
wherein a tip of the cone is centered in a middle of the dirty air inlet port.

4. The clean air system according to claim 3,
wherein the air intake fins are arranged around a periphery of an end of the cone opposite the tip of the cone.

5. The clean air system according to claim 4,
wherein the air intake fins extend from the periphery of the cone to an interior of the airflow chamber.

6. The clean air system according to claim 5,
wherein the air intake fins cause the air to flow through the airflow chamber in a vortex.

7. The clean air system according to claim 1,
wherein the UV light source is a UV bulb arranged in a center of the airflow chamber to allow air from the dirty air inlet port to be disinfected.

8. The clean air system according to claim 7, wherein the air circulates around the UV bulb in a vortex while being disinfected.

9. The clean air system according to claim 1,
wherein the UV light source comprises:
a plurality of UV light emitting diodes (LEDs),
wherein the plurality of LEDs are arranged in a grid pattern along an interior of the airflow chamber.

10. The clean air system according to claim 1, further comprising:
a fan located within or adjacent the dirty air inlet port for drawing dirty air into the dirty air inlet port and forcing air through the airflow chamber.

11. The clean air system according to claim 1,
wherein an inner diameter of the airflow chamber tapers from the dirty air inlet port to the clean air outlet port.

12. The clean air system according to claim 1,
wherein an inner diameter of the airflow chamber tapers from an end of the UV source to the clean air outlet port.

13. The clean air system according to claim 1,
wherein the dirty air inlet port is coupled to a plurality of table inlet ports arranged along a first section of a casino gaming table.

14. The clean air system according to claim 13,
wherein the clean air outlet port is coupled to a plurality of table outlet ports arranged around a second section of the casino gaming table.

15. The clean air system according to claim 14,
wherein the clean air system is coupled to an underside of the casino gaming table.

16. The clean air system according to claim 13,
wherein the first section has a semi-circular shape.

17. The clean air system according to claim 14, wherein the second section has a linear shape.

18. The clean air system according to claim 13,
wherein the table inlet ports are located along a side edge of the first section, and
wherein each table inlet port comprises:
an airflow opening;
a UV shield arranged adjacent the airflow opening;
an intake blade arranged adjacent and downstream from the UV shield.

19. The clean air system according to claim 18, wherein each table inlet port further comprises:
a UV light arranged adjacent the intake blade.
